(19) United States
(12) Patent Application Publication
(10) Pub. No.: US 2010/0114105 A1
(43) Pub. Date: May 6, 2010

(54) SYSTEM AND METHOD FOR VERTEBRAL INTERBODY FUSION

(75) Inventors: Joshua A. Butters, Chandler, AZ (US); Joseph Q. Marietta, Hyde Park, UT (US); Jason M. Glad, Lewiston, UT (US)

Correspondence Address: IMDS, INC., 124 SOUTH 600 WEST, LOGAN, UT, 84321, US

(73) Assignee: ROBERT REID,INC., Kita-ku (JP)

(21) Appl. No.: 12/262,824

(22) Filed: Oct. 31, 2008

(51) Int. Cl. A61B 17/58; A61F 2/44

(52) U.S. Cl. 606/99; 623/17.11; 623/17.16

(57) ABSTRACT

A surgical inserter and a fusion cage system which may provide for simplified fusion cage attachment and positioning. The inserter may comprise a collapsible collet with a locking rod. The fusion cage with a rectangular cross-section head may be secured to the inserter through the reverse tapered aperture of the fusion cage. The collet may be inserted into the reverse tapered aperture and the locking rod may control collapse of the collet. The collet may lock the fusion cage to the inserter allowing simplified manipulation of the collet with the inserter. The inserter with the attached fusion cage is inserted into a surgical patient. The fusion cage may be positioned within an intervertebral space. The locking rod may be withdrawn from the collet allowing collapse and withdrawal of the collet from the fusion cage leaving the fusion cage positioned between two vertebrae and the inserter withdrawn from the surgical patient.

### BACKGROUND OF THE INVENTION

1. The Field of the Invention

The present invention relates generally to the insertion of fusion cages, and more particularly, to systems and methods for implanting fusion cages with an inserter instrument for intervertebral disc replacement.

2. The Relevant Technology

Current cage inserter designs typically rely on a threaded connection between the fusion cage and the inserter. This connection type has its drawbacks, as the act of releasing the cage from the inserter requires a large number of revolutions to disengage the threaded member from the cage. This method of release is time consuming and the threaded hole needed in the cage causes high stress concentrations that can cause premature failure of the device. Additionally typical interfaces between the fusion cage and the inserter do not provide a truly locked configuration, allowing the fusion cage to rotate or become displaced while attached to the inserter.

Another technique utilizes an expandable collet inserter that provides a single-movement release that is superior to the threaded connection. However, the use of an expandable collet creates the possibility of exposing the cage attachment hole to large mechanical forces which can cause premature failure of the device.

As the above described instruments and techniques illustrate, the existing systems and procedures for inserting a fusion cage into intervertebral space, positioning of a cage between two vertebrae and releasing a cage may not be as effective as desired and may lead to further spinal surgeries.

### BRIEF DESCRIPTION OF THE DRAWINGS

Various embodiments of the present invention will now be discussed with reference to the appended drawings. It is appreciated that these drawings depict only typical embodiments of the invention and are therefore not to be considered limiting of its scope.

FIG. 1 illustrates a perspective slightly proximal side view of an intervertebral assembly comprising a fusion cage and an inserter;

FIG. 2 illustrates a perspective view of an inserter comprising a proximal handle portion and an insertion component with a collapsible collet protruding from a hollow outer shaft of the insertion component;

FIG. 3 illustrates a perspective view of the inserter of FIG. 2 with a toggle assembly in an outward position forcing the collapsible collet to protrude from the outer shaft and the locking sleeve in a locked outer position forcing the locking rod to protrude from the interference shaft controlling the collapsible collet;

FIG. 4 illustrates an exploded perspective view of the inserter of FIG. 2;

FIG. 5 illustrates a blown up distal end perspective view of the inserter of FIG. 2 with the collapsible collet within the hollow outer shaft;

FIG. 6 illustrates a blown up distal end perspective view of the inserter of FIG. 2 with the collapsible collet protruding from the hollow outer shaft;

FIG. 7 illustrates a blown up distal end perspective view of the inserter of FIG. 2 with a locking rod protruding from an interference shaft and the collapsible collet, controlling the collet, which is protruding from the hollow outer shaft;

FIG. 8 illustrates a perspective view of a fusion cage;

FIG. 9 illustrates a cross-sectional top view of the fusion cage of FIG. 8;

FIG. 10 illustrates a cross-sectional view of an alternate embodiment of a fusion cage;

FIG. 11 illustrates a cross-sectional side view of the interaction between the fusion cage and the distal end of the inserter and the collapsible collet with the locking rod in the unlocked position;

FIG. 12 illustrates a cross-sectional side view of the interaction between the fusion cage and the distal end of the inserter and the collapsible collet with the locking rod in the locked position;

FIG. 13 illustrates a cross-sectional side view of the interaction between the cage and the distal end of the inserter, the collapsible collet, the locking rod in a locked position with the collet engaging the fusion cage and locking the cage to the inserter.

FIG. 14 illustrates a cross-sectional side view of the distal end of the inserter with the locking rod and collapsible collet retracted and the cage released from the inserter.

### DETAILED DESCRIPTION OF THE PREFERRED EMBODIMENTS

The present invention relates to systems and methods for securing a fusion cage to and inserter and insertion of a fusion cage between two vertebrae. Those of skill in the art will recognize that the following description is merely illustrative of the principles of the invention, which may be applied in various ways to provide many different alternative embodiments. This description is made for the purpose of illustrating the general principles of this invention and is not meant to limit the inventive concepts in the appended claims.

One embodiment of the present invention includes a fusion cage and an inserter. A method of securing a fusion cage to an inserter consists of forcing a collapsible collet to protrude from an instrument body by pressing an actuation assembly to an outward position. The collet inserts into the proximal end of the fusion cage. A locking rod of the instrument is forced through the collet to control the collet from collapsing. The actuator assembly is pressed to an inward position forcing the collet to retract against a reverse tapered aperture, locking the fusion cage to the inserter.

A method for positioning a fusion cage between two vertebrae consists of securing a fusion cage to an inserter. The inserter inserts the fusion cage into the intervertebral space. Osteogenic substances such as harvested bone chips, bone growth factors, hydroxyapatite, tri calcium phosphate, bone growth factors, demineralized bone matrix or the like are packed into a fusion cage cavity that is accessible through a first and a second aperture. The inserter releases the fusion cage after placement within the intervertebral space.

The following definitions should be used with regard to this application:

A “reverse tapered aperture” means a hole that increases in diameter when moving from the outside of a device to the inside of a device, either continuously (e.g., a conical taper or the like) or discontinuously (one or more steps up in the diameter).

An “insertion component” means a long, slender, distal end of a surgical instrument that is intended to be introduced into the body of a surgical patient.

“Semi-tubular” means approximately half of a tube after a cut along the length of the tube.

“Snap into engagement” means an engagement in which parts come into a predetermined relative position or orientation via deformation of at least one of element, followed by relaxation of the deformation such that there is physical interference tending to resist motion of the parts from the predetermined relative position or orientation.

“Trigger” means a member that is shaped and sized to receive contact and pressure from one or more fingers of a user.

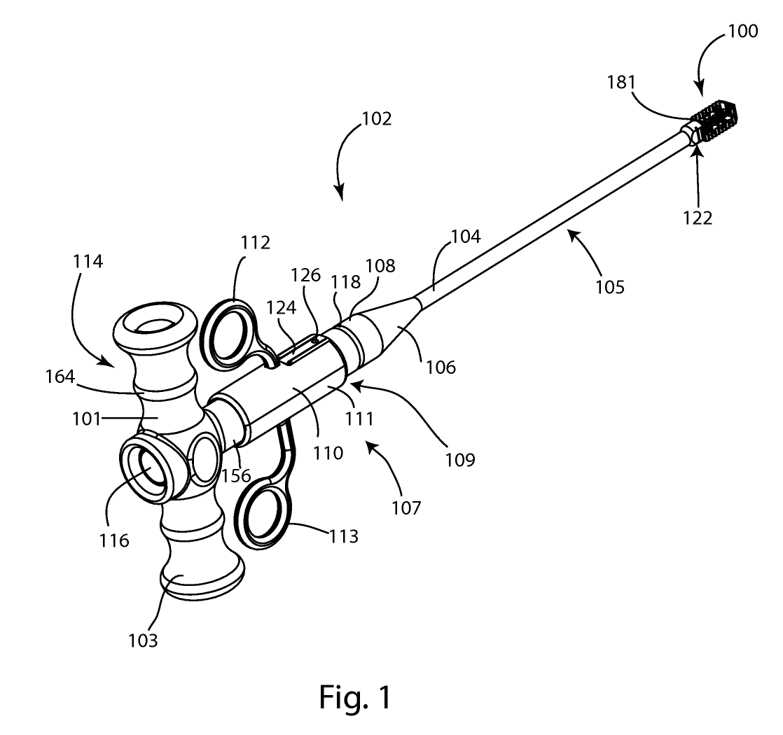

Referring to FIG. 1 a perspective view of an embodiment of a fusion cage 100 and an inserter 102 is shown. The inserter 102 includes a fusion cage interface 122 at a distal end allowing for a substantially complimentary fit with a proximal end 181 of the fusion cage 100. The proximal end of the inserter 102 within a proximal handle portion 114 includes an actuator button 116 which, when pressed, forces a collapsible collet 120 (not shown in FIG. 1) to protrude from the inserter distal end 122.

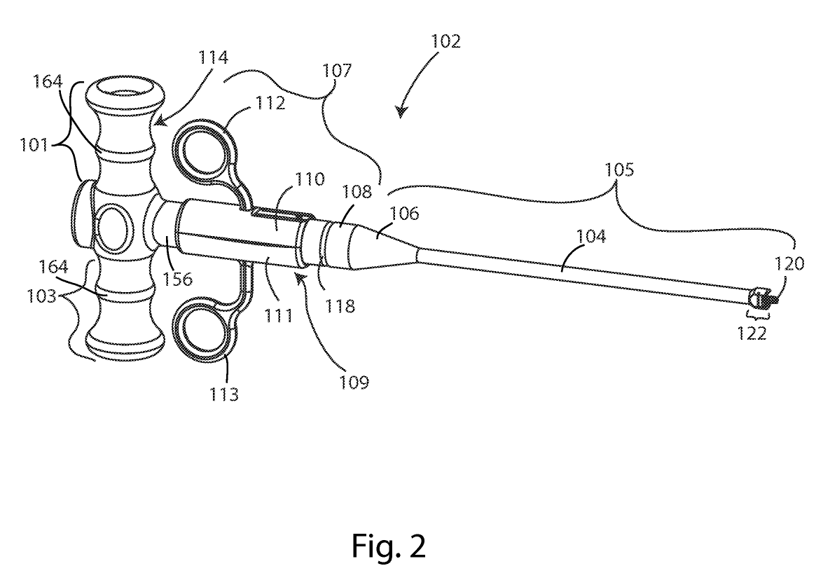

FIGS. 2-7 illustrate an embodiment of an inserter with a proximal handle portion, an intermediate portion, and an insertion component.

Referring to FIG. 2, the inserter 102 comprises the proximal handle portion 114 with a first grip 101 and a second grip 103 each extending non-parallel from the longitudinal axis with the second grip 103 extending opposite the first grip 101. The grips 101, 103 include handle grooves 164 which aid in the gripping of the proximal handle portion 114. An intermediate portion 107 of the inserter 102 comprises a proximal hollow outer shaft 108 which may be encompassed by a locking sleeve 109. The locking sleeve 109 comprises two semi-tubular portions comprising a first locking sleeve portion 110 and a second locking sleeve portion 111 with a first trigger 112 extending non-parallel from the first locking sleeve portion 110 and a second trigger 113 extending non-parallel from the second locking sleeve portion 111. Each locking sleeve portion 110, 111 comprises a feature configured to snap into engagement either in an unlocked snap groove 117 (not shown in FIG. 2), or a locked snap groove 118. The snap grooves 117, 118 are positioned on the exterior of the proximal hollow outer shaft 108 proximate a tapered shaft 106. The unlocked snap groove 117 is positioned proximate the locked snap groove 118 separated by some distance. The tapered shaft 106 is positioned at the distal end of the intermediate portion 107 of the inserter 102. The tapered shaft 106 decreases in size toward an insertion component 105 of the inserter 102.

An alternate embodiment (not shown) of the proximal handle portion may include a single grip extending in a single direction non-parallel from the longitudinal axis of the inserter. A further embodiment may also include a longer grip extending in a single direction non-parallel form the longitudinal axis of the inserter and a second shorter grip extending opposite the first grip.

An alternate embodiment for the locking sleeve (not shown) may include a single tubular component that encompasses an intermediate portion of the inserter. In addition the locking sleeve may include a single trigger (not shown) with a first trigger rod.

The insertion component 105 extends from the distal end of the tapered shaft 106 of the intermediate portion 107 along the same direction as the longitudinal axis of the intermediate portion 107. The insertion component comprises a generally circular hollow outer shaft 104 comprising an outer wall defining a bore, and an inserter distal end 122 which interacts with the fusion cage 100.

A collapsible collet 120 moves longitudinally independent from the hollow outer shaft 104. The collet 120 may maintain an open configuration or a collapsed configuration. The collet 120 may protrude from within the hollow outer shaft 104 of the insertion component 105 of the inserter distal end 122 when the actuator button 116 is pressed.

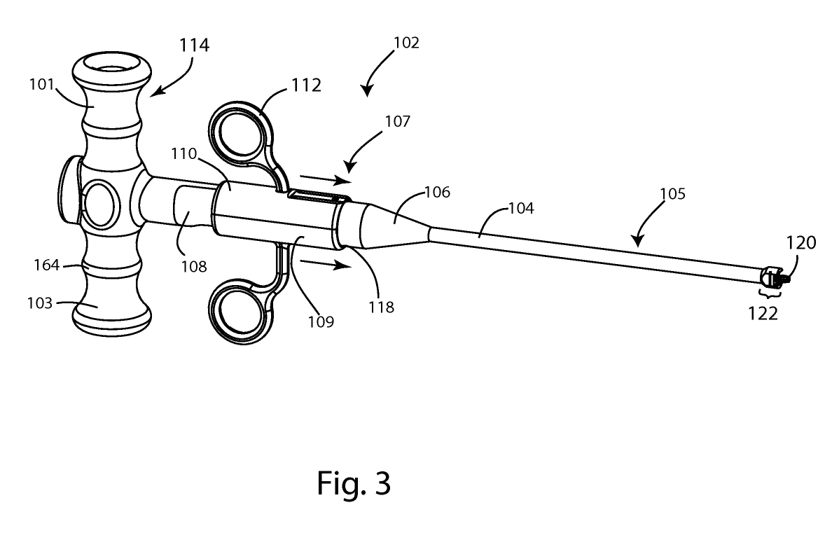

Referring to FIG. 3, the inserter 102 is shown with the locking sleeve 109 in a locked position snapped into engagement in the locked snap groove 118. Positioning the locking sleeve 109 in the locked position forces a locking rod 136 (not shown in FIG. 3) to protrude out of the inserter distal end 122 from within the insertion component 105 as well as protrude from within the collet 120, thereby controlling collapse of the collet 120.

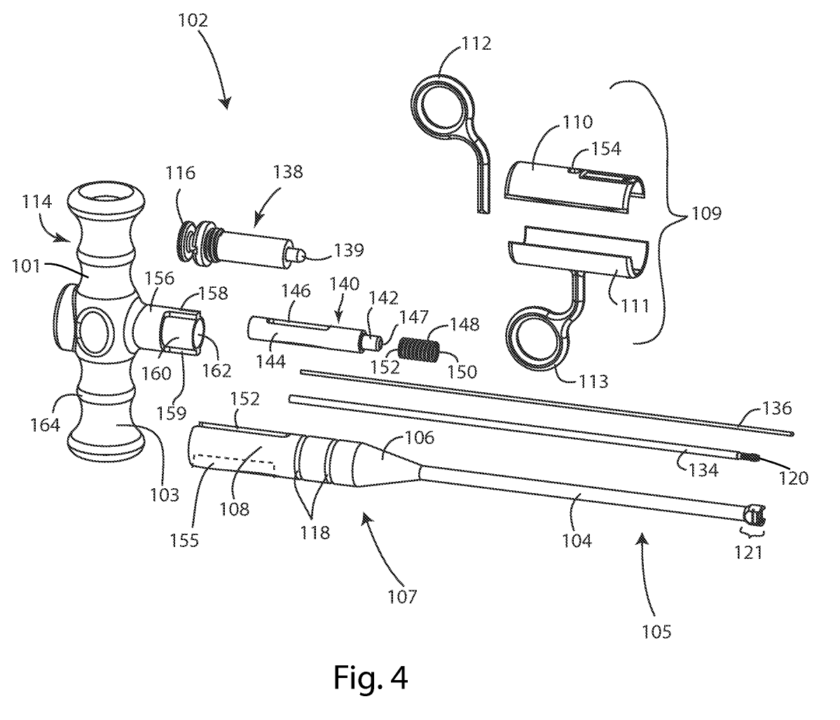

Referring to FIG. 4, the inserter 102 is shown in an exploded view exposing the individual parts. The proximal handle portion 114 further comprises a handle bore 162 extending lengthwise non-parallel to the handle grips 101, 103. An actuator assembly 138 passes at least partially through the handle bore 162. The proximal handle portion also includes an actuator shaft 156 which extends distally from the grips 101, 103 in a non-parallel fashion. The actuator shaft 156 includes a handle portion groove 160 and a handle portion rib 158 allowing for a slidable interface with the intermediate portion 107. More specifically the proximal hollow outer shaft 108 engages the handle portion groove 160 and an upper slot interface 153 of the intermediate portion 107 engages a first handle portion rib 158 and a lower slot interface 155 of the intermediate portion 107 engages a second handle portion rib 159. Each slot interface 153, 155 maintains a cavity between the proximal handle portion 114 and the intermediate portion 107 allowing for passage of a first trigger rod 115 through the intermediate portion 107. In the preferred embodiment the proximal handle portion 114 engages the intermediate portion by means of welding or securely fastened by some other means well known, such that once the proximal handle portion 114 and the intermediate portion 107 are engaged each cannot be separated from the other.

The actuator assembly 138 includes a toggle assembly within the actuator body. The distal end of the actuator assembly 138 comprises the actuator button 116. The proximal end of the actuator assembly 138 comprises an actuator dowel 139 which abuts a distal end of a plug 140.

The intermediate portion 107 includes the proximal hollow outer shaft 108 with the slot interface 153 proximal the unlocked snap groove 117 which is proximal the locked snap groove 118. The distal end of the intermediate portion includes the tapered shaft 106 decreasing in diameter toward the insertion component 105. The plug 140 and a spring 148 are shaped to pass at least partially through the proximal hollow outer shaft 108 substantially along the same longitudinal axis of the inserter 102. The plug 140 comprises a plug distal end 142 configured to abut a spring proximal end 152 of the spring 148. The plug distal end 142 also engages the proximal end of an interference shaft 134. The plug also includes an elongated plug body 144 which includes an elongated plug aperture 146 passing through the center of the plug body 144. The elongated plug aperture 146 passes through the plug body 144 in a non-parallel fashion in regard to the longitudinal axis of the inserter 102 or the plug 140. A plug bore 147 extends from the distal end of the plug 140 and passes through the plug 140 in communication with the elongated plug aperture 146.

The spring 148 also includes a spring distal end 150 configured to abut the surface of the interior of the tapered shaft 106. Abutment of the spring 148 against the plug distal end 142 and the interior of the tapered shaft 106 allows for compression of the spring 148 between the two surfaces.

The locking sleeve 109 at least partially encompasses the intermediate portion 107 with the first locking sleeve portion 110 and the second locking sleeve portion 111. The first locking sleeve portion includes a first locking sleeve aperture 154 that accepts the first trigger rod 115 of the first trigger 112. The first trigger rod 115 passes through the first locking sleeve aperture 154 which provides access to the upper slot interface 153. The first trigger rod 115 passes through the upper slot interface 153 which provides access to the elongated plug aperture 146. The first trigger rod 115 passes through the elongated plug aperture 146 which provides access to the lower slot interface 155. The first trigger rod 115 passes through the lower slot interface 153 engaging the second locking sleeve portion 111 at the point the second trigger 113 and second locking sleeve portion 111 connect.

The interference shaft 134 is hollow, comprising an interference shaft wall that defines an interference shaft bore. The interference shaft 134 is positioned at least partially within the hollow outer shaft 104 along a longitudinal direction. The distal end of the interference shaft 134 comprises the collapsible collet 120. A locking rod 136 is positioned at least partially within the interference shaft 134 along a longitudinal direction. The proximal end of the interference shaft 134 engages the plug distal end 142 passing longitudinally through the spring 148. The locking rod 136 passes through the plug bore 147 engaging the first trigger rod 115 which is positioned through the elongated plug aperture 146.

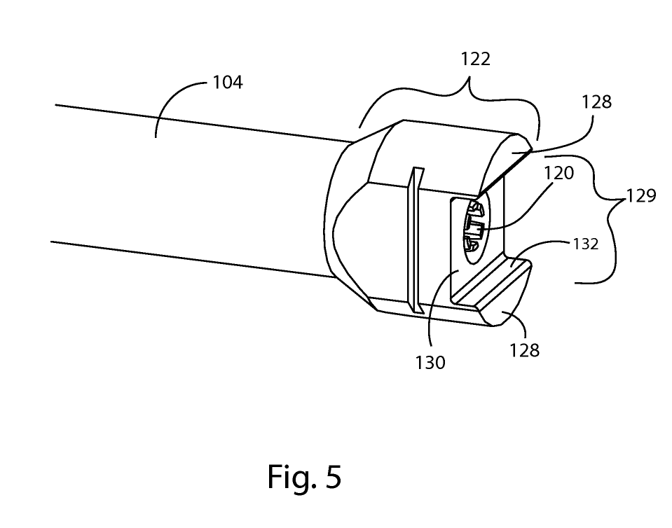

Referring to FIG. 5, the inserter distal end 122 includes an inserter distal wall 128 on opposite sides of an inserter trough 129 comprising a primarily rectangular cross sectional void with a trough base wall 130 and trough inner longitudinal walls 132. The inserter trough 129 facilitates attachment of the fusion cage 100 to the inserter. The collet 120 resides within the hollow outer shaft 104.

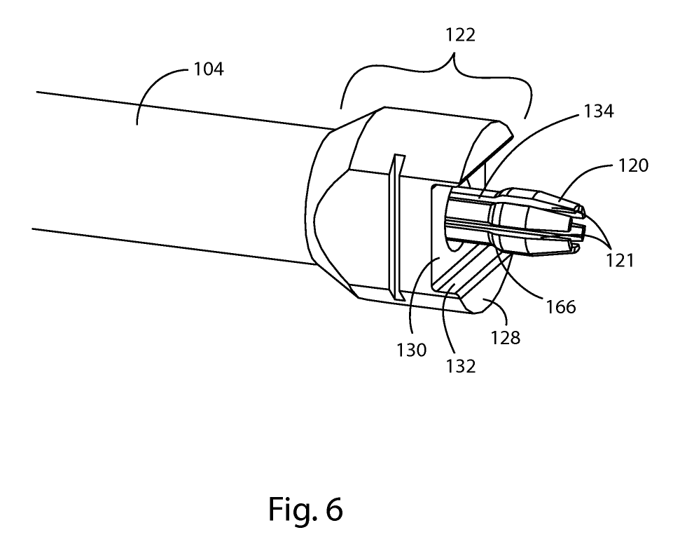

Referring to FIG. 6, the collet 120 includes collet grooves 121 allowing collapse of the collet and a collet proximal end 166 which tapers when moving proximally from the center of the collet 120. The collet 120 protrudes from within the hollow outer shaft 104 when the actuator button 116 is pressed extending the actuator dowel 139 to an outward position, pushing the plug 140 distally. The plug 140 moving distally moves the interference shaft 134 distally causing the collet 120 to protrude from the hollow outer shaft 104.

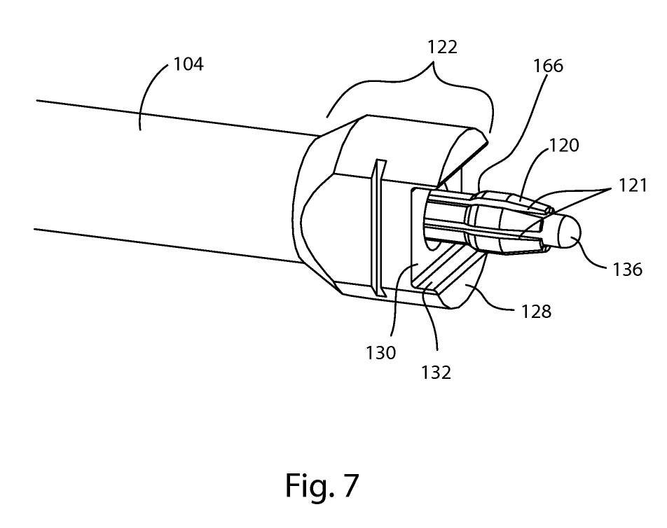

Referring to FIG. 7, the locking rod may protrude from within the interference shaft 134. The locking sleeve moves distally from an unlocked position to a locked position by pushing the triggers 112, 113 distally which forces the locking rod 136, connected to the first trigger rod, distally. The locking rod 136 controls the collet 120 from collapsing.

It is appreciated that the inserter 102 is comprised of many pieces and these pieces may be comprised of similar or many different materials. The inserter 102 and its pieces may be comprised of any metals, metal alloys, semi-rigid plastics or polyurethane. The interference shaft 134 and the collet 120 may be comprised of similar materials as the remainder of the inserter 102; however, the interference shaft 140 and the collet 120 may comprise metal, metal alloys and the like and may not consist of any elastomeric substances. It can be appreciated as well, that the pieces may also take on slightly different forms than depicted in the drawings such as straight, curved, bayoneted, offset, or the like, which assist in adapting the instrument for the access approach that the surgeon uses to get to the intervertebral space. Thus, the instruments of the present invention may be adapted to a variety of different surgical approaches.

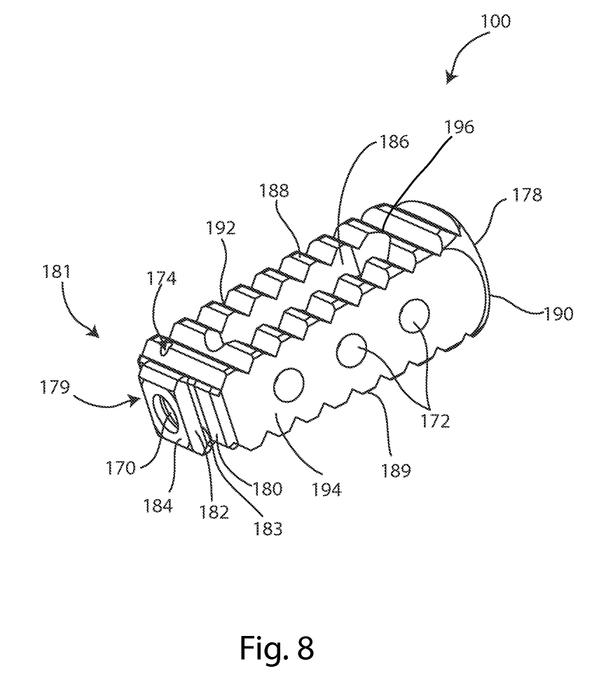

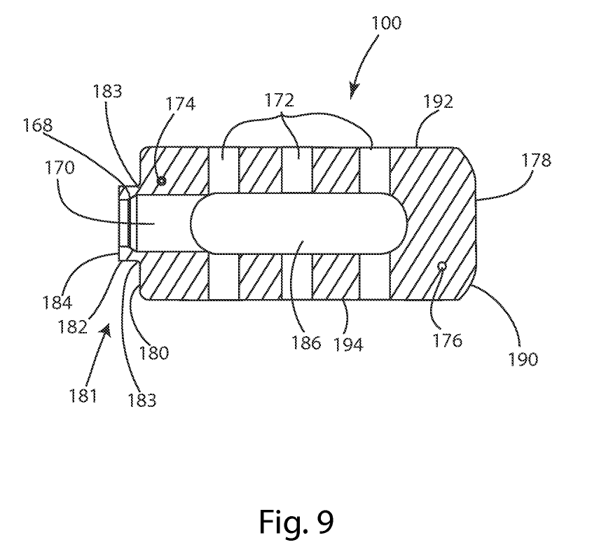

FIGS. 8 and 9 illustrate an embodiment of a fusion cage comprising a cavity, a distal wall, lateral walls and a proximal portion including a head with a rectangular cross section.

Referring to FIG. 8, the fusion cage 100 comprises an outer wall which includes a distal wall 178 which may be an uninterrupted surface, an inserter interface which comprises a head 179 with a generally rectangular cross section, the head 179 protrudes from a proximal wall 180, a shoulder 183 may be defined at the juncture of the head 179 and the proximal wall 180, a first corrugated vertebra engaging surface 188 and a second corrugated vertebra engaging surface 189 extending substantially parallel to one another from the proximal wall 180 to the distal wall 178. A first lateral wall 192 and a second lateral wall 194 are positioned opposite and parallel the first lateral wall 192, each lateral wall 192, 194 extending longitudinally from the proximal wall 180 to the distal wall 178. The distal wall 178 may include rounded edges or tapered edges at the junctures with the lateral walls 192, 194. A fusion cage aperture 196 may extend longitudinally along the fusion cage from the corrugated vertebrae engaging surfaces 188, 189. The lateral walls 192, 194 may include a plurality of pores 172. The pores 172 may be the same size or may vary in size. The fusion cage 100 also comprises a fusion cage cavity 186 located centrally between the lateral walls 192 and the proximal wall 180 and the distal wall 178, and in communication with the fusion cage aperture 196. Osteogenic substances may be inserted into the fusion cage cavity 186 through the fusion cage aperture 196 or through the pores 172.

Referring to FIG. 9, the head 179 of the fusion cage may comprise a generally circular reverse tapered aperture 170 which extends longitudinally from the proximal end of the fusion cage 100 to the fusion cage cavity 186. The reverse tapered aperture 170 comprises a collet engagement surface 168 that extends in a substantially diagonal or distal-lateral direction. The reverse tapered aperture 170 may receive the collet 120 of the inserter 102 facilitating attachment of the fusion cage 100 to the inserter 102. It can be appreciated that this reverse tapered aperture could take on many geometrical forms or engagement features to accomplish the same function of retaining the cage on the inserter via the collet. (e.g. a conical taper or stepped hole, etc) The fusion cage head 179 slides into the inserter trough 129 which may form a complimentary fit. A fusion cage head lateral wall 182 interfaces with the trough inner longitudinal walls 132 of the inserter as well as a fusion cage head base wall 184 interfaces with the trough base wall 130.

Positioning bores 174, 176 are present on the fusion cage 100 and may be used to determine if the fusion cage 100 has been placed in the correct location between two vertebrae. A proximal positioning bore 174 is located on the first corrugated vertebra engaging surface 188 toward the proximal end of the fusion cage 100 between the lateral wall 192 and the fusion cage cavity 186. A distal positioning bore 176 is positioned on the second corrugated vertebra engaging surface 189 toward the distal end of the fusion cage 100 between the opposite lateral wall 194 and the fusion cage cavity 186. Tantalum wire markers are inserted into the positioning bores so that x-rays of the fusion cage 100 within the intervertebral space can detect them to facilitate correct positioning of the fusion cage 100. However, it will be appreciated that any radio-opaque material or shape may also be used for insertion into the positioning bores 174, 176 to facilitate proper placement of the fusion cage 100 between two vertebrae.

The preferred embodiment of the fusion cage 100 is comprised of a carbon-fiber reinforced PEEK (polyetheretherketone). However, any biocompatible material may be used, including but not limited to stainless steels, titanium and its alloys, cobalt-chrome and its alloys, ceramics, polymers, biodegradable materials, allograft bone materials, or the like.

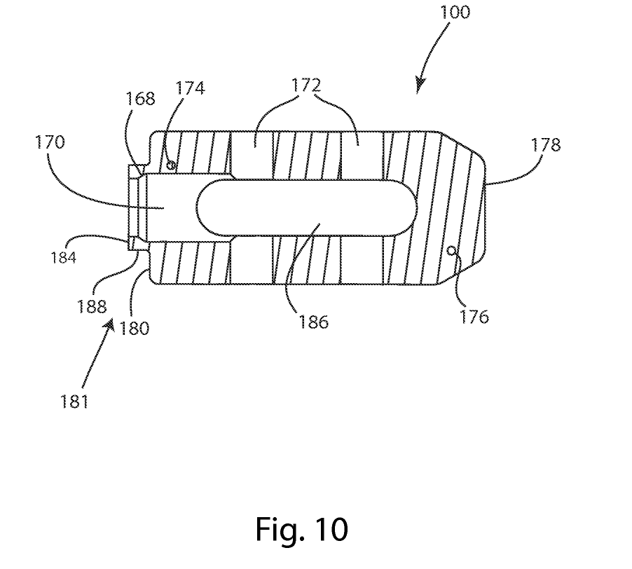

An alternate embodiment of a second fusion cage 200 is shown in FIG. 10. While most of the features remain constant with the previously disclosed fusion cage 100, the second fusion cage 200 may comprise fewer pores 172 while maintaining a plurality of pores, and the distal end 178 may taper more abruptly toward the lateral walls 192, 194. In addition the corrugated vertebrae engaging surfaces 188, 189 may include more or fewer ridges.

Another possible embodiment of the fusion cage may be a fusion cage wherein each lateral wall 192, 194 comprises a single pore (not shown). Further still another embodiment may include an aperture, a pore or other opening on the fusion cage distal wall 178 (not shown).

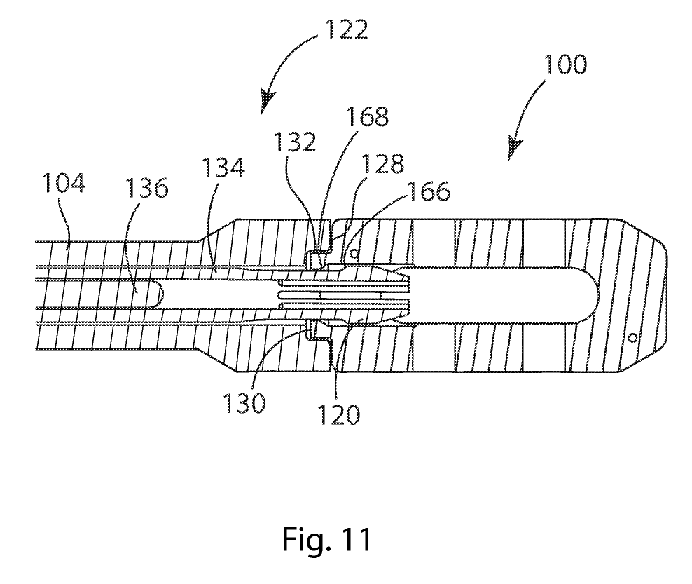

FIGS. 11 through 14 illustrate one method of implementing the present invention to attach a fusion cage to an inserter and to release a fusion cage from an inserter. Referring to FIG. 11, a fusion cage 100 is engaged with the inserter 102 at the inserter distal end 122. The fusion cage head 179 is slidably placed into the inserter trough 129. The collet 120 is forced distally by a user pressing the actuator button 116 which forces the toggle assembly to an outward position pushing actuator dowel 139 distally which forces the plug 140 to move distally compressing the spring 148 between the plug and the internal portion of the tapered shaft 106 which forces the interference shaft 134 distally causing the collet 120 to protrude from the inserter distal end 122, and more particularly, the collet 120 to protrude from within the inserter trough 129. The collet 120 is urged into a collapsed configuration as it is restricted by the reverse tapered aperture 170 to a smaller diameter as it is inserted into the fusion cage 120 reverse tapered aperture 170. The collet 120 may enter at least a portion of the fusion cage cavity 186. The collet 120 returns to its open configuration or original diameter after passing through the reverse tapered aperture 170.

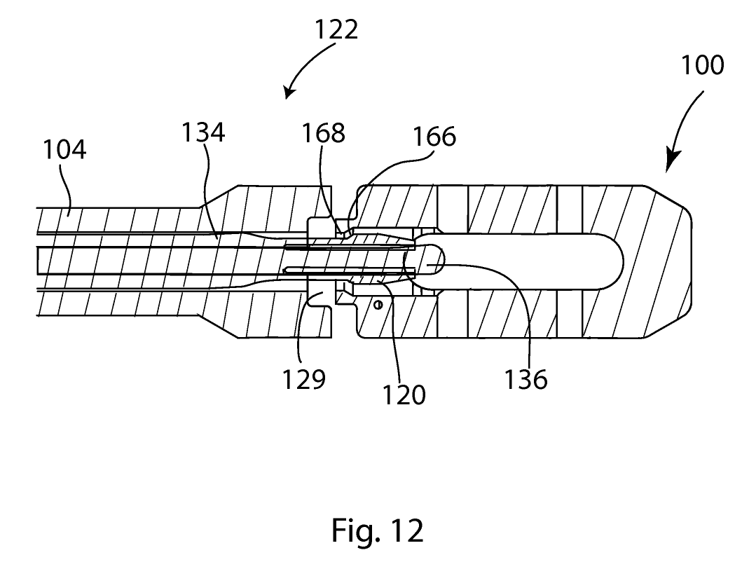

Referring to FIG. 12, the locking rod 130 protrudes distally from within the interference shaft 134 by a user thrusting the triggers 112, 113 distally which forces the locking sleeve from an unlocked position to a locked position (refer to FIG. 3). Forcing the triggers 112, 113 distally forces the locking sleeve 109 to unsnap from engagement from the unlocked snap groove 117 and snap into engagement into the locked snap groove 118. The first trigger rod 115 forces the locking rod 136 distally to protrude from the interference shaft 134 and more particularly from the collet 120. The locking rod 136 controls collapse of the collet 120.

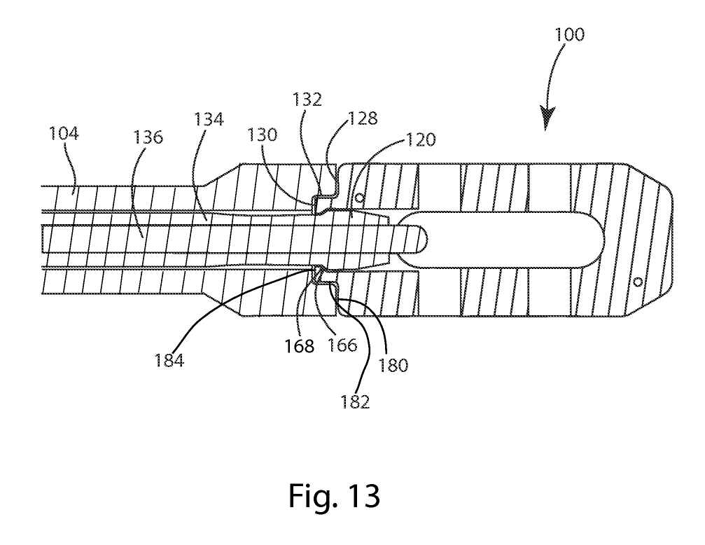

Referring to FIG. 13, a user presses the actuator button 116 which forces the toggle assembly to an inward position proximally retracting the actuator dowel 139 allowing the spring 148 to expand forcing the plug 140 distally. The collet 120 is retracted to engage the reverse tapered aperture 170. The locking rod controls collapse of the collet causing the collet proximal end 166 to engage the collet engagement surface 168 of the reverse tapered aperture 170 of the fusion cage 100. The retraction of the collet forces the fusion cage head 179 fully engage the inserter trough 129. The inserter distal wall 128 engages the fusion cage proximal wall 180. The trough base wall 130 engages the fusion cage head base wall 184 and the trough inner longitudinal walls 132 engage the fusion cage head lateral walls 182. The fusion cage 100 is incapable of being independently manipulated from the inserter 102 after the collet 120 engages the reverse tapered aperture 170 of the fusion cage 100.

The fusion cage 100 is positioned into the intervertebral space by a user manipulating the inserter 102. The vertebrae engaging surfaces 188, 189 may engage a superior and an inferior vertebra. Osteogenic substances may be inserted into the fusion cage cavity 186.

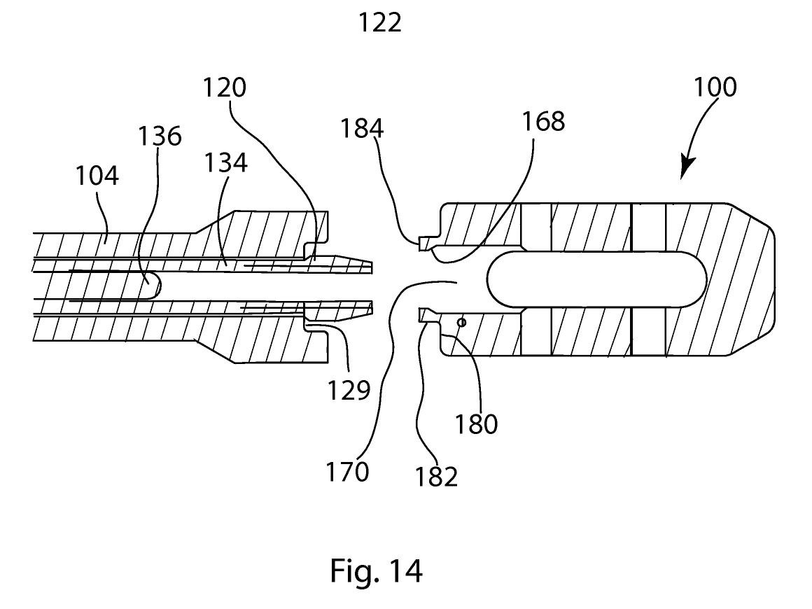

Referring to FIG. 14, release of the fusion cage 100 is accomplished by retraction of the locking rod 136 from the collet 120. A user pulls the triggers 112, 113 back from the locked position to the unlocked position wherein the locking sleeve 109 is unsnapped from engagement from the locked snap groove 118 and moves proximally and snaps into engagement in the unlocked snap groove 117. Retraction of the locking rod 136 allows uncoupling of the collet 120 from the fusion cage 100 and more particularly collapse of the collet 120 through the reverse tapered aperture 170. The spring 148 expands to its original size and presses against the interior tapered shaft 106 and the plug 140. The plug is forced proximally which retracts the interference shaft 134. The collet 120 collapses through the reverse tapered aperture 170 and retracts proximally into the hollow outer shaft 104. The fusion cage 100 remains frictionally in place between two vertebrae as the user withdraws the inserter 102 from a surgical patient.

The present invention may be embodied in other specific forms without departing from its spirit or essential characteristics. For example, above are described alternative examples of an inserter and fusion cages for spinal surgeries. It is appreciated that various features of the above-described examples of each can be mixed and matched to form a variety of other combinations and alternatives. It is also appreciated that this system should not be limited to a single method of use. This inserter and fusion cage attachment system may be used for any spinal surgery requiring fusion cage placement or replacement. As such, the described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

1. A system comprising:
a fusion cage sized to be inserted between a first vertebra and a second vertebra, the fusion cage comprising:
an outer wall comprising:
a distal portion; and
a proximal portion comprising:
a proximal wall; and
an inserter interface shaped to facilitate attachment of an inserter to the fusion cage, the inserter interface comprising a head with a rectangular cross section, the head protruding from the proximal wall to define a shoulder at a juncture with a proximal wall.

2. The system of claim 1, wherein the rectangular cross section comprises a square cross section.

3. The system of claim 1, wherein the head comprises a reverse tapered aperture sized to slidably receive a collet of the inserter.

4. The system of claim 1, wherein the outer wall further comprises:
a first corrugated vertebra engaging surface shaped to non-slidably engage the first vertebra; and
a second corrugated vertebra engaging surface shaped to non-slidably engage the second vertebra.

5. The system of claim 4, wherein the outer wall further comprises a first lateral wall having a first aperture sized to permit insertion of osteogenic substances into a cavity located centrally within the fusion cage or bony growth into the central cavity when the first and second corrugated vertebra engaging surfaces are positioned abutting the first and second vertebrae.

6. The system of claim 5, wherein the outer wall further comprises a second lateral surface having a second aperture sized to permit insertion of osteogenic substances into the cavity or bony growth into the cavity when the first and second corrugated vertebra engaging surfaces are positioned abutting the first and second vertebrae.

7. The system of claim 1, wherein the head comprises an aperture, the system further comprising the inserter, wherein the inserter comprises a recess having a rectangular cross section shaped to receive the head, and a collapsible collet insertable into an aperture of the head.

8. A system comprising:
a fusion cage inserter comprising:
a proximal handle portion; and
an insertion component comprising:
a hollow outer shaft comprising an outer wall defining a bore;
a hollow interference shaft positioned at least partially within the bore, the hollow interference shaft comprising an interference shaft wall defining an interference shaft bore and a collapsible collet positioned at a distal end of the interference shaft; and
a locking rod positioned at least partially within the interference shaft bore, wherein the locking rod is positionable to at least partially protrude from the interference shaft bore to control collapse of the collet.

9. The system of claim 8, wherein the outer wall comprises:
a fusion cage interface shaped to facilitate attachment of a fusion cage to the inserter, the fusion cage interface comprising a recess formed in a distal end of the outer wall, wherein the recess comprises a rectangular cross section shaped to slidably receive a rectangular head of the fusion cage.

10. The system of claim 8, wherein the collet is tapered such that the collet comprises a distal end wider than a proximal end of the collet such that the collet is shaped to engage a reverse tapered aperture within the fusion cage.

11. The system of claim 8, wherein the insertion component further comprises:
a plug within the proximal end of the insertion component, the plug comprising:
an elongated plug aperture engaging a proximal end of the hollow interference shaft; and
a distal end plug aperture slidably receiving the locking rod; and
a spring abutting a distal end of the plug.

12. The system of claim 11, wherein the handle portion houses an actuator assembly comprising:
a toggle assembly, wherein the toggle assembly is toggleable between an inward position and an outward position, wherein the inward position forces the collapsible collet to protrude from the hollow outer shaft and the outward position permits the collapsible collet to retract into the hollow outer shaft;
an actuator button; and
an actuator dowel protruding from the actuator assembly and engage a distal end of the plug.

13. The system of claim 12, wherein the actuator button abuts the distal end of the toggle assembly such that pressing the actuator button moves the toggle assembly to the outward position to force the actuator dowel to further protrude from the actuator assembly to push the plug and spring distally, thereby forcing the collet to protrude from the outer shaft.

14. A system comprising:
a fusion cage inserter comprising:
a proximal handle portion;
an insertion component comprising:
a collapsible collet;
an interference shaft; and
a locking rod at least partially within the interference shaft; and
a locking sleeve at least partially encircling a proximal portion of the insertion component, wherein the locking sleeve is slidable relative to the proximal handle portion to control coupling of the collet by moving the locking rod relative to the collet.

15. The system of claim 14, wherein the locking sleeve comprises:
a semi-tubular first locking sleeve member comprising a locking sleeve aperture; and
a first trigger comprising a first trigger rod, wherein the first trigger rod passes through the locking sleeve aperture, the insertion component and an elongated plug aperture.

16. The system of claim 15, wherein the locking sleeve further comprises:
a semi-tubular second locking sleeve member engaging the first trigger rod; and
a second trigger positioned on an opposite side of the locking sleeve from the first trigger.

17. The system of claim 15, wherein the first trigger rod engages the proximal end of the locking rod.

18. The system of claim 15, wherein the locking sleeve further comprises a semi-tubular second locking sleeve member; wherein the trigger rod passes through the insertion component and the elongated plug aperture and is attached to an interior surface of the second locking sleeve member.

19. The system of claim 14, wherein a distal end of the locking sleeve comprises a snap feature snapped into engagement with the insertion component.

20. The system of claim 19, wherein the snap feature is snapped into engagement with the locking sleeve at a locked position in which the locking rod extends into the collet to control withdrawal of the collet from an aperture of the fusion cage, and at an unlocked position displaced from the locked position, wherein in the unlocked position, the locking rod is displaced from within the collet to permit withdrawal of the collet from the aperture.

21. A method comprising:
positioning a fusion cage adjacent to an inserter;
coupling the fusion cage to the inserter, the fusion cage comprising an outer wall with a distal portion and a proximal portion comprising a proximal wall and an inserter interface comprising a head with a rectangular cross section, the head protruding from the proximal wall to define a shoulder at a juncture with a proximal wall; and
using the inserter to insert the fusion cage into an intervertebral space between a first vertebra and a second vertebra;
wherein coupling the fusion cage to the inserter comprises inserting the head into a recess formed in a distal end of the inserter, wherein the recess comprises a rectangular cross section.

22. The method of claim 21, wherein each of the rectangular cross sections comprises a square cross section.

23. The method of claim 21, wherein the head comprises a reverse tapered aperture, wherein coupling the fusion cage to the inserter further comprises inserting a collapsible collet of the inserter into the reverse tapered aperture.

24. The method of claim 21, wherein the outer wall further comprises a first corrugated vertebra engaging surface and a second corrugated vertebra engaging surface, wherein using the inserter to insert the fusion cage into the intervertebral space comprises:
non-slidably engaging the first vertebra with the first corrugated vertebra engaging surface; and
non-slidably engaging the second vertebra with the second corrugated vertebra engaging surface.

25. The method of claim 24, wherein the outer wall further comprises a first lateral wall having a first aperture, the method further comprising inserting an osteogenic substance into a cavity located centrally within the fusion cage through the first aperture after the first and second corrugated vertebra engaging surfaces have non-slidably engaged the first and second vertebrae.

26. A method comprising:
positioning a fusion cage adjacent to an inserter, the fusion cage comprising an aperture, the inserter comprising a proximal handle portion and an insertion component comprising a hollow outer shaft, a hollow interference shaft, and a locking rod;
coupling the fusion cage to the inserter; and
using the inserter to insert the fusion cage into an intervertebral space between a first vertebra and a second vertebra;
wherein the hollow outer shaft comprises an outer wall defining a bore;
wherein the hollow interference shaft is positioned at least partially within the bore and comprises an interference shaft wall defining an interference shaft bore and a collapsible collet positioned at a distal end of the interference shaft;
wherein coupling the fusion cage to the inserter comprises:
inserting the collapsible collet into the aperture; and
moving the locking rod into the collapsible collet to prevent collapse of the collapsible collet, thereby preventing withdrawal of the collapsible collet from the aperture.

27. The method of claim 26, wherein the outer wall comprises a fusion cage interface comprising a recess formed in a distal end of the outer wall, wherein the recess comprises a rectangular cross section; wherein the fusion cage comprises a rectangular head having a rectangular cross section; wherein coupling the fusion cage to the inserter further comprises receiving the rectangular head in the recess.

28. The method of claim 26, wherein the collet is tapered such that the collet comprises a distal end wider than a proximal end of the collet, wherein the aperture comprises a reverse tapered aperture.

29. The method of claim 26, wherein the insertion component further comprises a plug within the proximal end of the insertion component, the plug comprising an elongated plug aperture engaging a proximal end of the hollow interference shaft and a distal end plug aperture slidably receiving the locking rod; wherein the method further comprises:
sliding the plug to urge the hollow interference shaft to move such that the collapsible collet extends from the bore; and
deflecting a spring abutting the plug in response to sliding of the plug.

30. The method of claim 29, wherein the handle portion houses an actuator assembly comprising a toggle assembly, the toggle assembly comprising an actuator button and a actuator dowel protruding from a distal end of the toggle assembly; wherein sliding the plug comprises pressing an actuator button to toggle the toggle assembly from an outward position to an inward position in which the actuator dowel protrudes further to urge the plug to slide distally.

31. A method comprising:
positioning a fusion cage adjacent to an inserter, the fusion cage comprising an aperture, the inserter comprising a proximal handle portion, a locking sleeve, and an insertion component comprising a collapsible collet, a hollow interference shaft, and a locking rod;
coupling the fusion cage to the inserter; and
using the inserter to insert the fusion cage into an intervertebral space between a first vertebra and a second vertebra;
wherein the locking sleeve at least partially encircles a proximal portion of the insertion component;
wherein coupling the fusion cage to the inserter comprises:
inserting the collapsible collet into the aperture; and
sliding the locking sleeve relative to the proximal handle portion to couple the collet to the fusion cage by moving the locking rod relative to the collet.

32. The method of claim 31, wherein the locking sleeve comprises:
a semi-tubular first locking sleeve member comprising a locking sleeve aperture; and
a first trigger comprising a first trigger rod, wherein the first trigger rod passes through the locking sleeve aperture, the insertion component and an elongated plug aperture.

33. The method of claim 32, wherein the locking sleeve further comprises:
a semi-tubular second locking sleeve member engaging the first trigger rod; and
a second trigger positioned on an opposite side of the locking sleeve from the first trigger.

34. The method of claim 32, wherein the first trigger rod engages the proximal end of the locking rod.

35. The method of claim 32, wherein the locking sleeve further comprises a semi-tubular second locking sleeve member; wherein the trigger rod passes through the insertion component and the elongated plug aperture and is attached to an interior surface of the second locking sleeve member.

36. The method of claim 31, wherein a distal end of the locking sleeve comprises a snap feature snapped into engagement with the insertion component.

37. The method of claim 36, wherein the snap feature is snapped into engagement with the locking sleeve at a locked position in which the locking rod extends into the collet to control withdrawal of the collet from an aperture of the fusion cage, and at an unlocked position displaced from the locked position, wherein in the unlocked position, the locking rod is displaced from within the collet to permit withdrawal of the collet from the aperture.
